(19) United States
(12) Patent Application Publication
(10) Pub. No.: US 2026/0061424 A1
(43) Pub. Date: Mar. 5, 2026

(54) CARTRIDGE WITH LIQUID REAGENTS FOR AUTOMATIC CHEMISTRY ANALYSIS OF RECREATIONAL WATER INCLUDING POOLS AND SPAS

(71) Applicant: ZODIAC POOL SYSTEMS LLC, Carlsbad, CA (US)

(72) Inventors: Timothy McNeil, Carlsbad, CA (US); Anthony Levacque, Carlsbad, CA (US); Mark J. Bauckman, San Marcos, CA (US); Hendrikus Johannes van der Meijden, Glen Austin (ZA); Taylor Kuter, Carlsbad, CA (US); Abian van der Meijden, Glen Austin (ZA)

(73) Assignee: ZODIAC POOL SYSTEMS LLC, Carlsbad, CA (US)

(21) Appl. No.: 19/308,405

(22) Filed: Aug. 25, 2025

Related U.S. Application Data
(60) Provisional application No. 63/688,746, filed on Aug. 29, 2024.

(51) Int. Cl. B01L 3/00; G01N 33/18; G01N 35/00; G01N 35/10

(52) U.S. Cl. CPC B01L 3/52; G01N 33/18; G01N 35/00732; G01N 35/1002; B01L 2200/025; B01L 2200/0689; B01L 2200/16; B01L 2300/123

(57) ABSTRACT

Systems and methods for monitoring water of a swimming pool or spa include containers or cartridges with one or more liquid reagents for automatic chemistry analysis of recreational water. The cartridges provide a store or supply of one or more reagents and may be quickly and easily replaced and replenished by a user. The cartridges may be self-sealing when the cartridge is not engaged with the monitoring device.

### REFERENCE TO RELATED APPLICATION

This application claims the benefit of and priority to U.S. Provisional Ser. No. 63/688,746, filed on Aug. 29, 2024, and entitled CARTRIDGE WITH LIQUID REAGENTS FOR AUTOMATIC CHEMISTRY ANALYSIS OF RECREATIONAL WATER INCLUDING POOLS AND SPAS, the content of which is hereby incorporated by reference in its entirety.

### FIELD OF THE INVENTION

The invention relates to water testing or monitoring systems, and more particularly, but not necessarily exclusively, to water testing or monitoring systems for swimming pools or spas.

### BACKGROUND

Maintaining water quality is important for swimming pools, spas, hot tubs, and other water containing vessels (hereinafter “swimming pools or spas”) to avoid issues for users of the swimming pool or spa as well as equipment of the swimming pool or spa. For example, if the water chemistry of the swimming pool or spa is off, a health hazard may be posed to users and/or operation of various pool equipment and/or systems may be compromised.

Conventional user-controlled approaches for monitoring water quality include chemistry kits, remote testing, and maintenance service calls. Conventional chemistry kits may be complicated and not user-friendly to pool owners, and the individual using the chemistry kit may be uncertain of the results. Remote testing requires taking a sample of water to a store or chemistry laboratory to analyze the sample but may pose issues of sample contamination, improper storage, and/or a change in water chemistry during transit. Service calls rely on the availability of maintenance personnel and further rely on the expertise of the person to properly conduct water quality testing and to properly understand and interpret the results from such testing. Moreover, reagents utilized in such user-controlled approaches are considered consumables, and a supply of the reagent must be on hand and periodically replenished to provide continued monitoring. Such reagents may be susceptible to spoilage over time, and thus may not be in a good condition by the time the user attempts to utilize the reagent, thereby providing inaccurate results. Moreover, reagents could leak or drip on the person, clothes, equipment, etc. As such, many existing approaches to monitoring water quality may be inaccurate, inconvenient, labor-intensive and time-consuming.

In addition to user-controlled approaches, other approaches for monitoring water quality have included probes and floating devices, but these other approaches also suffer from various deficiencies. For example, probes may drift and age over time, often requiring extensive maintenance for cleaning and calibration of the probes. In addition, it may be difficult to predict or troubleshoot an incorrect measurement from a probe, and the incorrect measurement may lead to over-dosing or under-dosing of chemicals into the pool. Floating devices may become stuck in non-desirable locations and may be removed from the pool by any user, thereby providing incorrect measurements or compromising the performance of the device. In addition, floating devices are battery-operated, thereby requiring periodic maintenance to ensure proper powering of such devices.

U.S. Pat. No. 7,988,916 to Bremauer (“Bremauer”) describes “an apparatus for precisely mixing and/or analysing small volumes of fluids for optic values. ” Bremauer at col. 1, ll. 8-10. According to Bremauer, the “apparatus for measuring a range of small volumes” includes “a) a single reaction chamber, b) a reciprocatable piston in said chamber, c) a first inlet to said chamber . . . d) at least one further inlet . . . e) a sealable outlet . . . f) said piston is operable within said chamber to selectively and precisely vary the internal volume of said chamber . . . g) said first inlet has a first valve selectively operable... until a predetermined volume of said first fluid is drawn into said chamber . . . h) said second inlet has a second valve selectively operable . . . to progressively draw said second fluid into said chamber until a predetermined condition is met . . . [and] j) said outlet is sealed by an outlet valve located in said base adjacent the internal surface of said chamber. ” Bremauer at col. 3, ll. 33-67.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, an aerial device system includes an adjustable railing and a control system. The adjustable railing is positionable around at least a portion of a base platform of the aerial device system. The adjustable railing is movable between a stowed position and a deployed position. A height of the adjustable railing in the deployed position is greater than a height of the adjustable railing in the stowed position. The control system is operably coupled to the adjustable railing and is configured to control the positioning of the adjustable railing. In various embodiments, the control system may detect or receive a position of a component of an aerial device of the aerial device system as being in a stowed device position or a deployed device position and detect or receive a condition of a secondary device of the aerial device system. In certain embodiments, the control system may position the adjustable railing in the deployed position based on the component of the aerial device having the stowed device position and based on the condition of the secondary device having a predefined target condition. In various embodiments, the control system may position the adjustable railing in the stowed position based on at least one of the component of the aerial device having the deployed device position or the condition of the secondary device not having the predefined target condition.

According to certain embodiments, an aerial device system includes an adjustable railing and a control system. The adjustable railing is positionable around at least a portion of a base platform of the aerial device system and is movable between a stowed position and a deployed position. The control system is operably coupled to the adjustable railing for controlling the positioning of the adjustable railing. In certain embodiments, the control system may detect or receive a position of a component of an aerial device of the aerial device system, detect or receive a condition of a secondary device of the aerial device system, and position the adjustable railing in the deployed position or the stowed position based on the position of the component of the aerial device and based on the condition of the secondary device.

According to certain embodiments, a method of controlling a cab guard safety system includes detecting or receiving a position of a component of an aerial device relative to a base platform of an aerial device system, detecting or receiving a condition of a secondary device of the aerial device system, and positioning, by a control system, an adjustable railing to a deployed position or a stowed position based on the detected position of the component of the aerial device and based on the detected condition of the secondary device.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The specification makes reference to the following appended figures, in which use of like reference numerals in different figures is intended to illustrate like or analogous components.

FIG. 1 illustrates a pool system with a water monitoring system having a cartridge system according to embodiments.

FIG. 2 illustrates the cartridge system of FIG. 1 according to embodiments.

FIG. 3 is another view of the cartridge system of FIG. 1 according to embodiments.

FIG. 4 is another view of the cartridge system of FIG. 1 according to embodiments

FIG. 5 is a sectional view of the cartridge system of FIG. 1 according to embodiments.

FIG. 6 is another sectional view of the cartridge system of FIG. 1 according to embodiments.

FIG. 7 illustrates a cartridge interface of the cartridge system of FIG. 1 according to embodiments.

FIG. 8 illustrates a cartridge of the cartridge system of FIG. 1 according to embodiments.

FIG. 9 is a sectional view of the cartridge of FIG. 8 according to embodiments.

FIG. 10 is an exploded view of the cartridge of FIG. 8 according to embodiments.

FIG. 11 illustrates a cover of the cartridge of FIG. 8 according to embodiments.

FIG. 12 illustrates a base of the cartridge of FIG. 8 according to embodiments.

FIG. 13 is a sectional view of a portion of the base of FIG. 12 according to embodiments.

FIG. 14 illustrates a spout of the cartridge of FIG. 8 according to embodiments.

FIG. 15 is a sectional view of the spout of FIG. 14 with a sealing member.

FIG. 16 is a sectional view of a portion of the cartridge assembly of FIG. 1 according to embodiments.

FIG. 17 illustrates the spout and sealing member of FIG. 15 with a spout support according to embodiments.

FIG. 18 illustrates the cartridge system of FIG. 1 with a cover of the water monitoring system of FIG. 1 according to embodiments.

FIG. 19 illustrates the cartridge removed from the cover of FIG. 18 according to embodiments.

FIG. 20 is a sectional view of the cartridge system and cover of FIG. 18.

### DETAILED DESCRIPTION

Described herein are systems and methods for monitoring water of a swimming pool or spa. More particularly, described herein are containers or cartridges with one or more liquid reagents for automatic chemistry analysis of recreational water, such as but not limited to water of swimming pools and spas.

In certain embodiments, the systems and methods described herein may provide a store or supply of one or more reagents, which may be used by a monitoring system for automatic measurement of one or more water parameters, which may be quickly and easily replaced and replenished by a user. In certain embodiments, the systems and methods described herein may provide simple and/or quick replacement of a cartridge while minimizing and/or eliminating the risk of installing the cartridge in an incorrect position or orientation on the monitoring device, thereby minimizing and/or eliminating the potential of mixing reagents and/or providing the incorrect reagent for a particular water parameter measurement.

In various embodiments, the systems and methods described herein may provide cartridges which are self-sealing (e.g., leak-proof, or substantially leak-proof) when the cartridge is not engaged with the monitoring device, thereby minimizing and/or eliminating leakage of the reagents. As used herein, “self-sealing” refers to the ability of the cartridges to automatically seal, and does not necessarily mean that the cartridge is completely sealed from air and/or does not mean that air is present within the container. Additionally, or alternatively, the self-sealing nature of the cartridge may facilitate storage of the reagents when not in use, thereby improving the quality and life of the reagents for use by an end user.

In some embodiments, the systems and methods described herein optionally may include an oxygen scavenger and/or an oxygen absorber within the cartridge to help remove and/or decrease the level of oxygen within the cartridge, thereby reducing and/or eliminating oxidization of the liquid reagents to ensure that the liquid reagents are fresh and in good condition during usage.

Various other benefits and advantages may be realized with the systems, devices, and methods provided herein, and the aforementioned advantages should not be considered limiting.

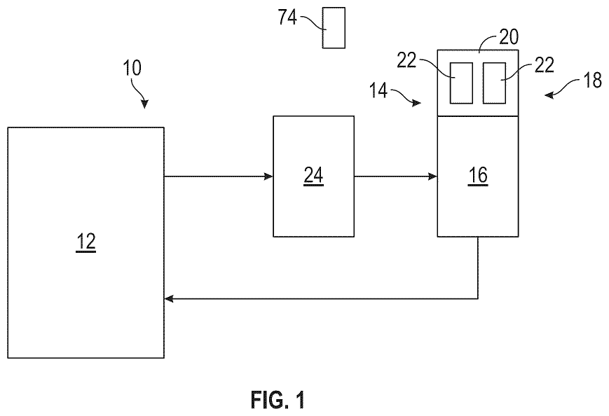

FIG. 1 illustrates an example of a pool system 10 according to embodiments. The pool system 10 generally includes a swimming pool or spa 12 (hereinafter “pool 12”) and a monitoring system 14 for measuring one or more water parameters of water of the pool 12. As non-limiting examples, the monitoring system 14 may measure water parameters such as but not limited to pH, free chlorine, total chlorine, total alkalinity, cyanuric acid concentration, and/or other parameters. In some embodiments, the monitoring system 14 may measure one water parameter, although in other embodiments, the monitoring system 14 may measure a plurality of water parameters, such as two water parameters, three water parameters, four water parameters, five water parameters, and/or more than five water parameters. As discussed in detail below, the monitoring system 14 may include means for drawing and mixing water and a reagent within a testing chamber and means for measuring a water parameter of the mixed water within the testing chamber.

The monitoring system 14 generally includes a monitoring device 16 and a cartridge system 18. The cartridge system 18 generally includes a cartridge 20 with one or more stores 22 of liquid reagent. The number of stores 22 of liquid reagent should not be considered limiting. In some embodiments, and as illustrated in FIG. 5, each store 22 of liquid reagent may include a container 23 with a spout 25 (only one container 23 is illustrated for clarity of the figure).

In some embodiments, the containers 23 optionally are constructed from a relatively flexible material such that the containers 23 have an adjustable shape and/or may at least partially conform to the volume of liquid reagent container therein. As non-limiting examples, the containers 23 may be flexible packaging constructed from various types of plastics, such as but not limited to polyethylene (PE), polypropylene (PP), polyamide, polyvinyl chloride (PVC), and polyethylene terephthalate (PET), from metal or foil laminates, from semi-rigid plastics, combinations thereof, and/or other materials as desired. In one non-limiting example, the containers 23 are flexible pouches. In a non-limiting example, the containers 23 are flexible pouches constructed from a metal or foil laminate. Optionally, containers 23 constructed from a metal or foil laminates may help remove and/or decrease the level of oxygen within the cartridge, thereby reducing and/or eliminating oxidization of the liquid reagents to ensure that the liquid reagents are fresh and in good condition during usage. In other embodiments, the containers 23 need not be constructed from a flexible material and instead may be constructed from a relatively rigid material which maintains its shape when empty.

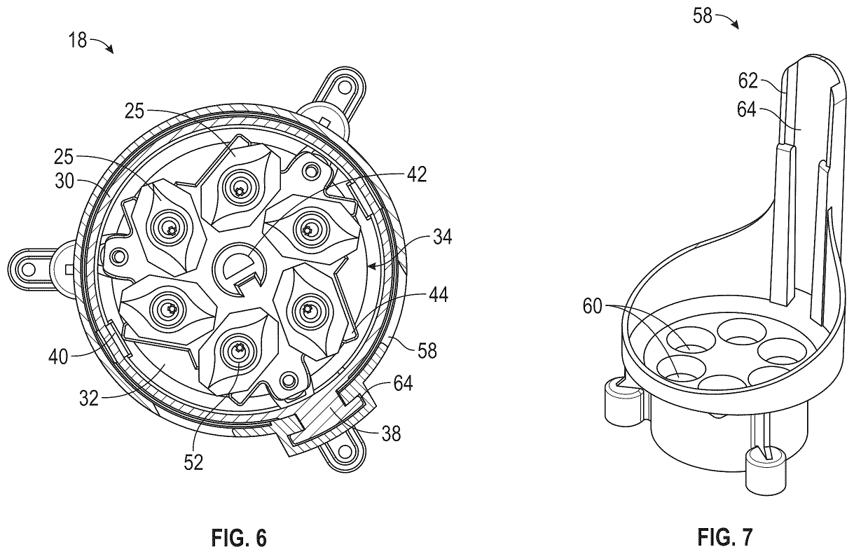

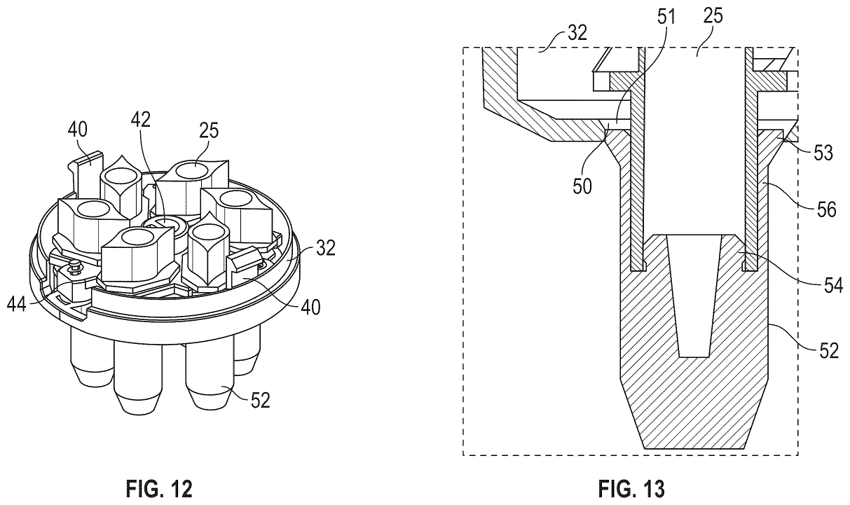

In certain embodiments, and as represented by the spouts 25 in FIGS. 6 and 12, for example, the containers 23 (e.g., pouches and/or other suitable containers) may be arranged in a circular and/or circumferential manner relative to a center of the cartridge 20. Optionally, and as represented by the spouts 25, the containers 23 may extend radially from the center of the cartridge 20, further optionally inclined such that the containers 23 extend both radially and circumferentially relative to the center of the cartridge. In certain embodiments, the arrangement of the containers 23 within the cartridge 20 may be provide a compact and efficient storage of a plurality of reagents, thereby improving performance and functionality of the monitoring system 14.

In various embodiments, the cartridge 20 is attachable to and detachable from the monitoring device 16 such that the supply of liquid reagent to the monitoring device 16 may be replaced and/or removed as otherwise desired. As non-limiting examples, FIGS. 18 and 19 illustrate the cartridge 20 attached to a cover 66 of the monitoring device 16 (FIG. 18) and removed from the cover 66 of the monitoring device 16 (FIG. 19). In this example, the cover 66 includes a mounting recess 68 which may at least partially receive the cartridge 20 and/or provide support and/or stability to the cartridge 20 when the cartridge 20 is installed with the monitoring device 16. In other embodiments, various other devices, mechanisms, and/or systems may be utilized to attach the cartridge 20 to the monitoring device 16.

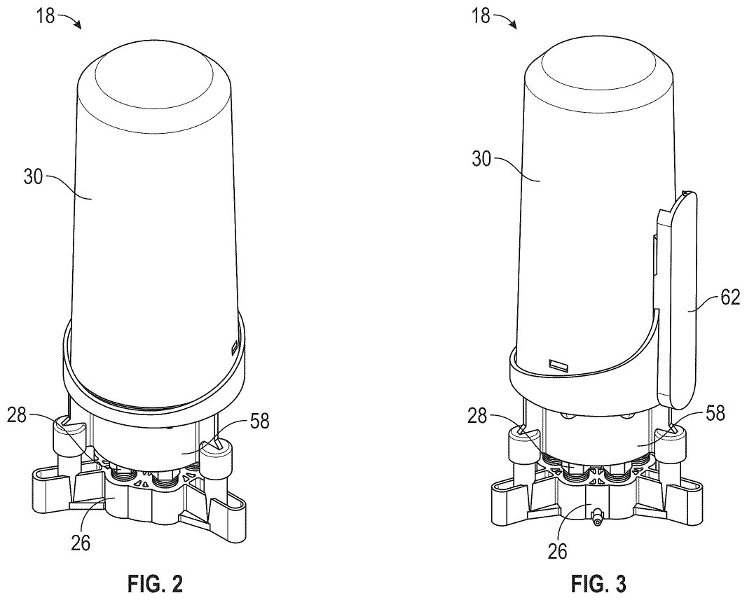

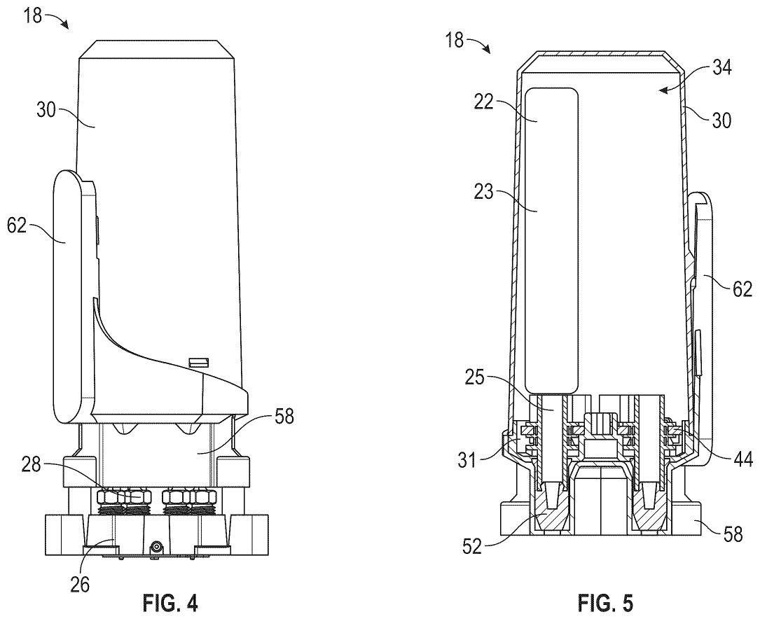

When the cartridge 20 is coupled with the monitoring device 16, the monitoring device 16 may be fluidly connected to the one or more stores 22 of liquid reagent such that the monitoring device 16 may utilize the liquid reagent(s) for measuring one or more water parameters of water of the pool 12. In various embodiments, and as illustrated in FIGS. 2-4, the cartridge system 18 may be engaged with a monitoring device interface 26 of the monitoring device 16. In some embodiments, the monitoring device interface 26 may include one or more fluid connectors 28 for engaging and receiving the reagent of the cartridge system 18. In the embodiment illustrated, the fluid connectors 28 are dispensing needles. However, in other embodiments, any type of fluid connector 28 may be utilized as desired.

In certain embodiments, the monitoring system 14 may be provided inline with one or more systems or equipment of the pool system 10. As an example, and as illustrated in FIG. 1, the monitoring system 14 may be provided inline with a pump 24 of a water circulation system. In other embodiments, the monitoring system 14 may be provided inline at other locations and/or relative to other equipment of the pool system 10 as desired.

FIGS. 2-20 illustrate the cartridge system 18 in greater detail.

Referring to FIGS. 5 and 8-13, for example, in various embodiments the cartridge 20 includes a cover 30 and a base 32 attachable to the cover 30. The cover 30 defines an internal volume 34 for receiving the one or more stores 22 of liquid reagent. In the embodiment illustrated, the internal volume 34 is configured to receive five stores 22 of liquid reagent; however, in other embodiments, the internal volume 34 may receive any number of stores 22 as desired, and the particular shape of the cover 30 and/or internal volume 34 should not be considered limiting. In some non-limiting examples, the cover 30 may be constructed from a relatively rigid material capable of maintaining its shape while empty. As non-limiting examples, the cover 30 may be constructed from materials such as but not limited to various metals, ceramics, composites, plastics, and/or other materials as desired. In such embodiments, the cover 30 constructed from a relatively rigid material may provide protection to the liquid reagent stored therein. Optionally, the cover 30 may be constructed from a relatively rigid material while the containers 23 are constructed from a relatively flexible or non-rigid material (and/or are otherwise flexible containers 23).

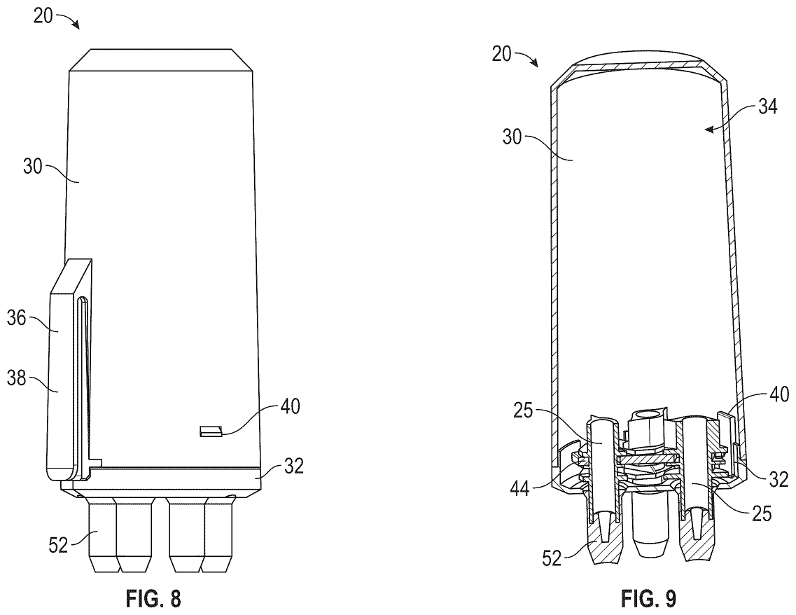

Optionally, and as best illustrated in FIGS. 8, 10, and 11, the cover 30 includes one or more alignment features 36, which may be utilized to position the cover 30 in a predefined orientation or configuration relative to the monitoring device 16 such that reagents within the cover 30 are properly aligned with corresponding ports of the monitoring device 16. In some embodiments, the alignment feature 36 of the cover 30 may cooperate with a corresponding alignment feature 62 of the monitoring device 16 and/or the cartridge system 18 as discussed in detail below. As a non-limiting example, the alignment feature 36 is configured to engage a corresponding alignment feature 62 of the cartridge interface 58. In this example, the alignment feature 36 is generally a ridge 38 receivable within a groove 64 as the alignment feature 62. However, in other embodiments, the groove 64 may be on the cover 30 and the ridge 38 may be on the cartridge interface 58. Moreover, in other embodiments, other cooperating alignment features may be utilized as desired, such as but not limited to pins, grooves, channels, stoppers, hooks, clips, clasps, geometries, combinations thereof, and/or as otherwise desired, and as such, the particular alignment features illustrated as well as the location and number of alignment features should not be considered limiting.

The base 32 of the cartridge 20 may be attachable to and detachable from the cover 30 using various techniques or mechanisms as desired and such that the internal volume 34 may be selectively closed or open. In the embodiment illustrated, snap-fit connectors 40 on the base 32 engage corresponding apertures 41 on the cover 30; however, other features or combinations of features may be utilized as desired.

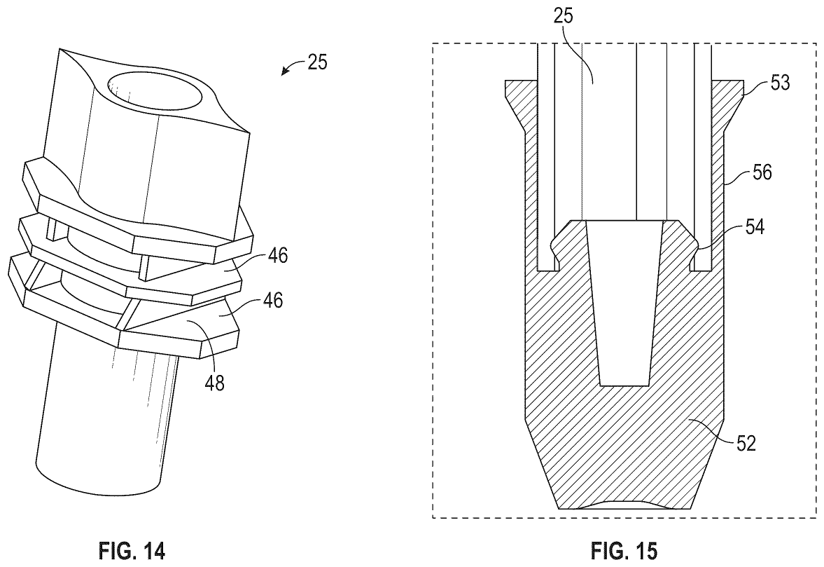

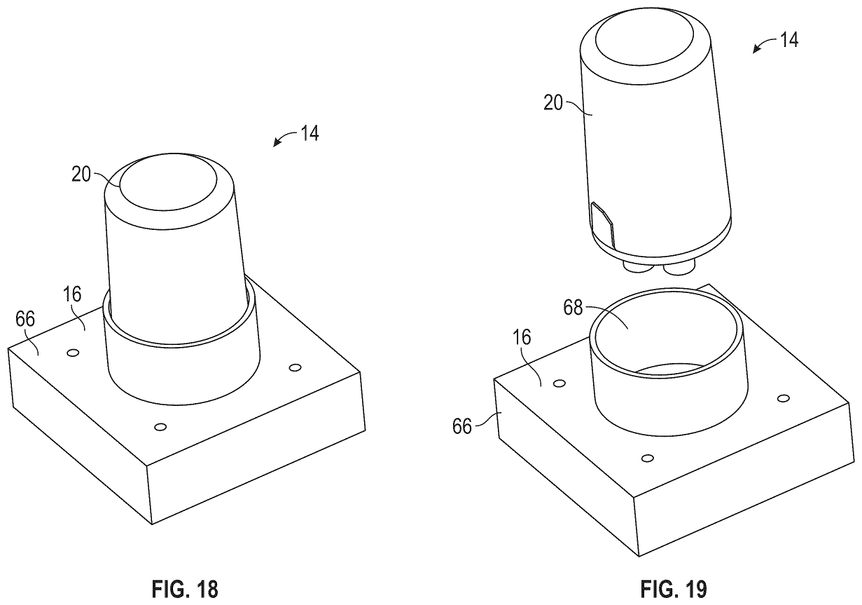

As best illustrated in FIGS. 8, 10, and 12, the base 32 may include a center support 42, which may position and/or support a spout support 44. The spout support 44 may engage and/or support the spout 25 of each container 23 within the internal volume. Optionally, and as illustrated in FIGS. 14 and 17, one or more spouts 25 may include height positioning features 46, such as but not limited to one or more slots 48, which may be engaged by the spout support 44 to position the spouts 25 (and thus containers 23) at different heights relative to the base 32.

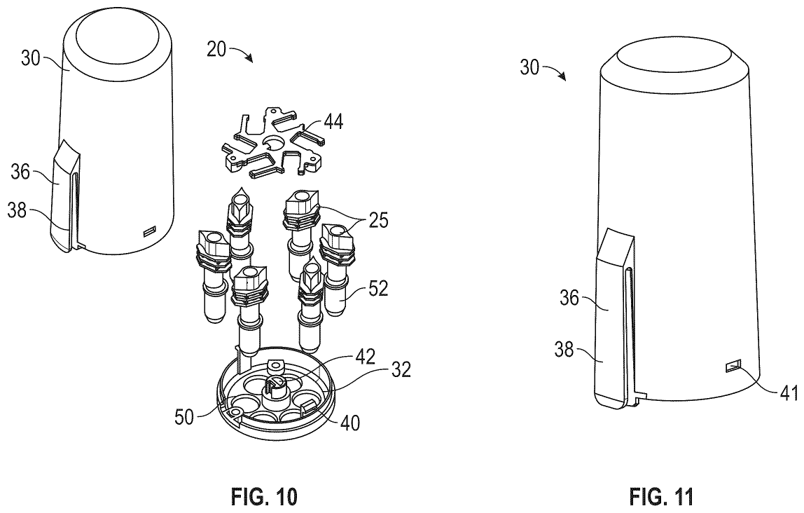

In various embodiments, and as best illustrated in FIG. 10, the base 32 includes a plurality of base apertures 50, each of which may at least partially receive a corresponding spout 25 (see, e.g., FIG. 13). In some embodiments, each spout 25 optionally includes a flow control device or mechanism as part of the container 23 for controlling a flow of the reagent from a particular spout 25 and/or through the corresponding base aperture 50. In other embodiments, and as illustrated in FIGS. 10, 12, 13, 15, and 16, the cartridge 20 additionally or alternatively includes one or more sealing members 52 for controlling a flow of reagent and/or sealing the spouts 25. In certain embodiments, the one or more sealing members 52 are self-sealing, thereby minimizing and/or eliminating leakage or flow of reagent from the containers 23 in the absence of engagement with a fluid connector 28. Sealing members 52 may be constructed from various materials as desired, such as but not limited to various synthetic or natural rubbers as desired. In one non-limiting example, the sealing members 52 may be constructed from silicone.

As illustrated in FIGS. 10, 12, and 13, for example, in certain embodiments the sealing members 52 are at least partially supported within a corresponding base aperture 50. Optionally, the base aperture 50 includes a tapered surface 51 configured to engage a flange 53 of the sealing member 52 and thereby position the sealing member 52 within the base aperture 50. However, in other embodiments, the tapered surface 51 and/or flange 53 may be omitted and/or the sealing member 52 may be positioned relative to the base aperture 50 using other features, mechanisms, techniques, and/or as otherwise desired.

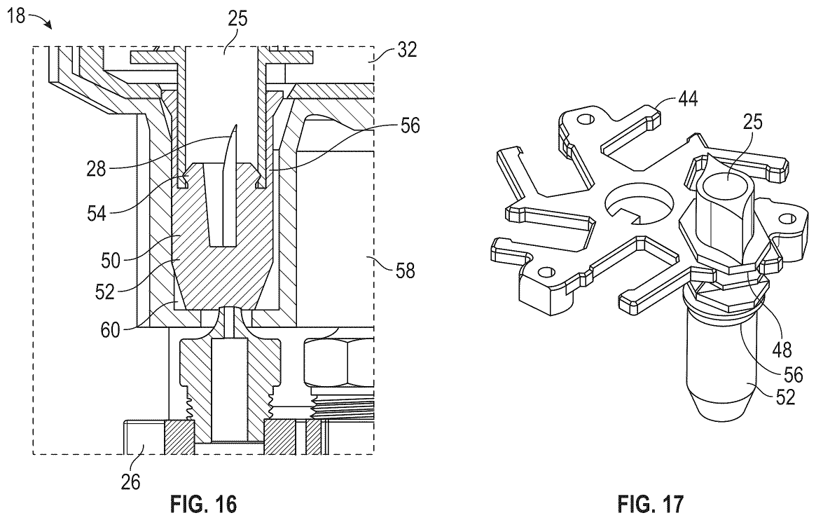

In various embodiments, and as illustrated in FIG. 15, each sealing member 52 includes an inner wall 54 and an outer wall 56. Optionally, a height of the inner wall 54 is less than a height of the outer wall 56, although it need not be in other embodiments. In certain embodiments, a portion of the spout 25 may be received within the sealing member 52 between the inner and outer walls 54, 56 such that the inner wall 54 is at least partially received within the spout 25 and the outer wall 56 at least partially surrounds the spout 25. In various embodiments, such an arrangement may allow the sealing member 52 to form a seal with the spout 25. In certain embodiments, and as illustrated in FIG. 16, the fluid connector 28 may be received within the inner wall 54 to establish a fluid connection between the particular container 23 and the monitoring device 16. In certain embodiments, the sealing member 52 forms a seal around the fluid connector 28, and in the absence of the fluid connector 28, the sealing member 52 self-seals, thereby sealing the container 23. In other embodiments, sealing members 52 utilized with the cartridge 20 may have other shapes, configurations, and/or combinations of features as desired, and the particular sealing members illustrated should not be considered limiting.

Referring to FIGS. 2-7, in addition to the cartridge 20, the cartridge system 18 may include a cartridge interface 58. In certain embodiments, the cartridge interface 58 may be attached to the monitoring device 16 (optionally at the monitoring device interface 26), and the cartridge 20 is attachable to and detachable from the cartridge interface 58.

In certain embodiments, the cartridge interface 58 may include one or more cavities 60 (see, e.g., FIG. 7) corresponding to the locations of the fluid connectors 28. When the cartridge 20 is engaged with the cartridge interface 58, the spouts 25 and/or sealing members 52 may be positioned within corresponding cavities 60 to engage corresponding fluid connectors 28. In certain embodiments, the cartridge interface 58 optionally includes one or more alignment features 62 for engaging the one or more alignment features 36 of the cover 30 such that the cartridge 20 is assembled with the cartridge interface 58 (and thus monitoring device 16) in a predefined orientation. In certain embodiments, such alignment may prevent and/or minimize cross-contamination of reagents and/or may ensure that correct reagents are utilized for a particular measurement. In the embodiment illustrated, the one or more alignment features 62 include a groove 64 configured to receive the ridge 38. However, as previously discussed, the particular alignment features illustrated should not be considered limiting, and other alignment features may be utilized as desired and at other locations and/or in other numbers of alignment features as desired.

Optionally, the cartridge system 18 with the cartridge 20 may include additional and/or alternative locking features for controlling the ability of the cartridge 20 to connect with the monitoring device, the cartridge interface, and/or other components as desired. In one non-limiting example, such locking features may be physical features, such as but not limited to features that are keyed and/or include complimentary mating or engaging features. Additionally, or alternatively, the locking features may include mating features such as but not limited to electronic mating features. As a non-limiting example, the monitoring device, cartridge interface, and/or other component may include a mating area, and the cartridge 20 may include a communication tag (such as but not limited to an NFC tag) which may allow for connection between the cartridge 20 and the monitoring device (or other) when properly engaged. Other locking features or combinations of features may be utilized as desired, and the aforementioned examples should not be considered limiting.

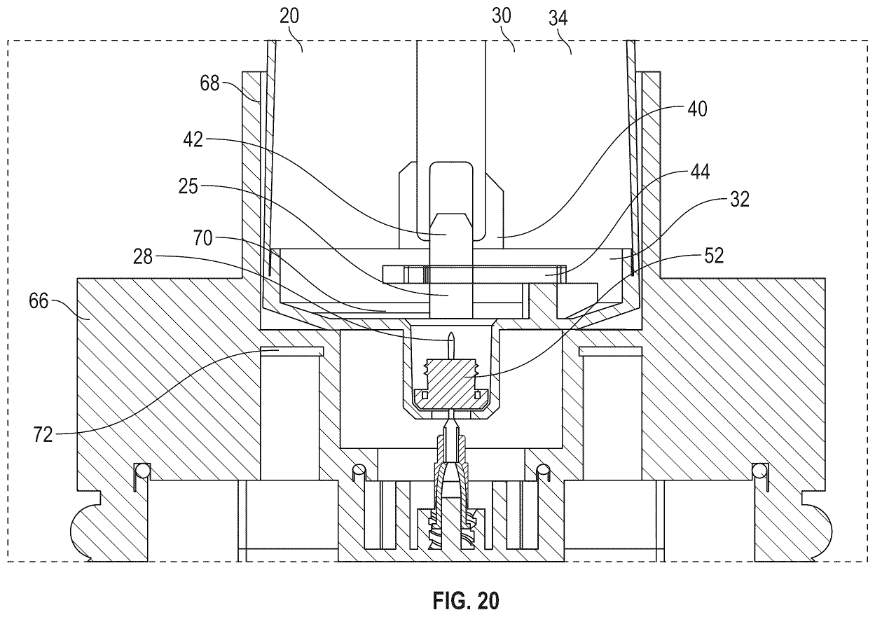

Referring to FIG. 20, in some embodiments, the cartridge system 18 may include an identifier or tag 70 or other suitable device or mechanism which may allow for information to be obtained without requiring a dedicated power source. As a non-limiting example, the tag 70 may be an NFC tag. The tag 70 may be provided at various locations on the cartridge 20 as desired, such as but not limited to the cover 30, the base 32, combinations thereof, and/or as otherwise desired. Information provided by the tag 70 may be various information related to the cartridge 20 and/or the cartridge system 18, such as but not limited to a reagent expiration date, a manufacture date, an identification of the type and/or arrangement of reagents within the cartridge 20, reagent source information, installation instructions, troubleshooting instructions, combinations thereof, and/or as otherwise desired.

In certain embodiments, and as illustrated in FIG. 20, the monitoring system 14 optionally may include one or more cartridge sensors 72 for monitoring and/or detecting at least an installation status of the cartridge 18 relative to the monitoring device 16. As non-limiting examples, the cartridge sensor 72 may detect that the cartridge 18 is installed using various sensing techniques such as detecting a presence, a pressure, a proximity, an amount of light, magnetic field, combinations thereof, and/or other sensing techniques as desired.

In some embodiments, a control system 74 (e.g., processor and/or memory) of the monitoring system 14 (see FIG. 1) may perform various actions and/or generate various control responses based on a detection that the cartridge 18 is installed or removed from the monitoring device 16 by the cartridge sensor 72. As a non-limiting example, because some reagents may degrade over time and/or have an expiration date, based on a new detection of the installation of the cartridge 18, the control system 74 may initiate a reagent timer whereby upon expiration of the timer, the control system 74 may send an alert or prompt a user to refill and/or replace the cartridge 18. The reagent time may be various durations of time as desired, and in some cases may depend on the reagents within the cartridge 18. As a non-limiting example, the control system 74 may start a reagent timer of 3 months upon a detection of a new installation of a cartridge 18. Additionally, or alternatively, a control response of the control system 74 may include controlling components of the monitoring device 16 based on a detection that the cartridge 18 is installed or removed from the monitoring device 16. As a non-limiting example, based on a detection by the cartridge sensor 72 that the cartridge 18 has been removed from the monitoring device 16, the control system 74 may halt, limit, prevent, and/or otherwise control operation of components of the monitoring device 16 such as a valve, a plunger, etc. Conversely, in this example, based on a detection by the cartridge sensor 72 that the cartridge 18 is installed with the monitoring device 16, the control system 74 may enable operation of components of the monitoring device 16 such as a valve, a plunger, etc. Various other control responses may be implemented as desired, and the aforementioned examples are for illustrative purposes only.

The cartridge system 18 with the cartridge 20 having one or more liquid reagents for automatic chemistry analysis of recreational water may allow simple and quick replacement of the cartridge without the risk of installing it in a wrong position (e.g., to avoid mixing reagents and using the wrong reagent for a measurement). The cartridge 20 may further minimize and/or avoid leakage of reagents by utilizing the self-sealing sealing members 52, further facilitating use by an end user. Various other benefits and advantages may be realized with the systems, devices, and methods provided herein, and the aforementioned advantages should not be considered limiting.

Exemplary concepts or combinations of features of the invention may include:
A. A cartridge with one or more liquid reagents for automatic chemistry analysis of recreational water including swimming pools and spas, the cartridge comprising resealable or self-sealing ports for installing and/or removing the cartridge with a monitoring device without leaking the one or more liquid reagents.
B. A monitoring system comprising:
   i. a monitoring device configured to measure one or more water parameters using one or more liquid reagents; and
   ii. a cartridge containing a supply of the one or more liquid reagents, wherein the cartridge is attachable to and detachable from the monitoring device, wherein the supply of the one or more liquid reagents comprises a plurality of containers arranged circumferentially.
C. A cartridge comprising a rigid outer cover defining an internal volume and one or more flexible or non-rigid reagent containers within the internal volume, each of the one or more reagent containers configured to store a supply of or more liquid reagents for automatic chemistry analysis of recreational water including swimming pools and spas.
D. A cartridge system comprising:
   i. a cartridge interface attachable to a monitoring device of a monitoring system for automatic chemistry analysis of recreational water including swimming pools and spas; and
   ii. a cartridge configured to store a supply of one or more liquid reagents, wherein the cartridge is removable from the cartridge interface and is engageable with the cartridge interface at a predetermined orientation using one or more alignment features, the cartridge comprising one or more flexible containers for the one or more liquid reagents.
E. A cartridge system comprising:
   i. a cartridge interface attachable to a monitoring device of a monitoring system for automatic chemistry analysis of recreational water including swimming pools and spas, the cartridge interface comprising a plurality of reagent ports; and
   ii. a self-sealing cartridge configured to store a supply of one or more liquid reagents, wherein the cartridge comprises a predefined orientation such that a particular liquid reagent is supplied to a predefined one of the plurality of reagent ports.
F. A cartridge system comprising:
   i. at least one of:
      i. a monitoring device; or
      ii. a cartridge interface attachable to the monitoring device of a monitoring system for automatic chemistry analysis of recreational water including swimming pools and spas,
      iii. wherein the monitoring device or the cartridge interface comprises a mating area; and
   ii. a cartridge configured to store a supply of one or more liquid reagents, wherein the cartridge comprises a communication tag.
G. The cartridge of any preceding or subsequent statements or combination of statements, wherein the communication tag is an NFC tag.
H. The cartridge of any preceding or subsequent statements or combination of statements, wherein the cartridge is self-sealing.
I. The cartridge of any preceding or subsequent statements or combination of statements, wherein the reagent containers comprise flexible or non-rigid containers.
J. The cartridge of any preceding or subsequent statements or combination of statements, wherein the reagent containers comprise flexible pouches.
K. The cartridge of any preceding or subsequent statements or combination of statements, wherein each reagent container comprises a spout, and wherein the cartridge further comprises a sealing member for each spout configured to automatically seal the spout.
L. The cartridge of any preceding or subsequent statements or combination of statements, further comprising a sealing member for each spout of each reagent container, wherein the sealing member comprises an outer wall and an inner wall, wherein the spout is received between the outer wall and the inner wall, and wherein:
   i. the outer wall is configured to form a seal the spout; and
   ii. the inner wall is configured to form a seal with the spout.
M. The cartridge of any preceding or subsequent statements or combination of statements, wherein the sealing member is configured to receive a fluid connector of a monitoring device within the inner wall to fluidly connect the reagent container with the monitoring device.
N. The cartridge of any preceding or subsequent statements or combination of statements, wherein the alignment features comprise at least one ridge and at least one slot.
O. The cartridge of any preceding or subsequent statements or combination of statements, wherein the cartridge comprises an outer cover and a base with one or more apertures corresponding to the one or more liquid reagents.
P. The cartridge of any preceding or subsequent statements or combination of statements, wherein a height of each reagent container within the internal volume is adjustable.
Q. The cartridge of any preceding or subsequent statements or combination of statements, further comprising a spout support within the internal volume.
R. A method comprising:
   i. attaching a cartridge with one or more liquid reagents for automatic chemistry analysis of recreational water including swimming pools and spas to a monitoring device, the cartridge comprising the one or more liquid reagents in one or more flexible containers; and
   ii. detaching the cartridge from the monitoring device.
S. The cartridge of any preceding or subsequent statements or combination of statements, wherein containers for the liquid reagent are in a circular pattern or arrangement.
T. The cartridge of any preceding or subsequent statements or combination of statements, further comprising a communication tag configured to provide information without a dedicated power supply.
U. The cartridge of any preceding or subsequent statements or combination of statements, wherein the reagent containers comprise a metal or foil laminate.
V. The cartridge of any preceding or subsequent statements or combination of statements, wherein the sealing member comprises silicone.

These examples are not intended to be mutually exclusive, exhaustive, or restrictive in any way, and the invention is not limited to these example embodiments but rather encompasses all possible modifications and variations within the scope of any claims ultimately drafted and issued in connection with the invention (and their equivalents). For avoidance of doubt, any combination of features not physically impossible or expressly identified as non-combinable herein may be within the scope of the invention. Further, although applicant has described devices and techniques for use principally with swimming pools or spas, persons skilled in the relevant field will recognize that the present invention conceivably could be employed in connection with other water containing vessels and in other manners, particularly but not limited to underwater installations. Finally, references to “pools” and “swimming pools” herein may also refer to spas or other water containing vessels used for recreation, training, or therapy.

That which is claimed:

1. A cartridge comprising:
one or more liquid reagents for automatic chemistry analysis of recreational water including swimming pools and spas, wherein containers for the liquid reagents are in a circular pattern or arrangement; and
resealable or self-sealing ports for installing and/or removing the cartridge with a monitoring device without leaking the one or more liquid reagents.

2. A cartridge system comprising the cartridge of claim 1 and a cartridge interface attachable to a monitoring device of a monitoring system for automatic chemistry analysis of recreational water including swimming pools and spas, wherein the cartridge is removable from the cartridge interface and is engageable with the cartridge interface at a predetermined orientation using one or more alignment features.

3. The cartridge of claim 1, further comprising a communication tag configured to provide information without a dedicated power supply.

4. The cartridge of claim 3, wherein the communication tag comprises an NFC tag.

5. The cartridge of claim 1, wherein each reagent container comprises a spout, and wherein the cartridge further comprises a sealing member for each spout configured to automatically seal the spout.

6. The cartridge of claim 1, wherein the sealing member comprises silicone.

7. The cartridge of claim 1, wherein the reagent containers comprise flexible or non-rigid containers.

8. A cartridge system comprising:
a cartridge interface attachable to a monitoring device of a monitoring system for automatic chemistry analysis of recreational water including swimming pools and spas, the cartridge interface comprising a plurality of reagent ports; and
a self-sealing cartridge configured to store a supply of one or more liquid reagents, wherein the cartridge comprises a predefined orientation such that a particular liquid reagent is supplied to a predefined one of the plurality of reagent ports.

9. The cartridge system of claim 8, wherein the cartridge further comprises a communication tag configured to provide information without a dedicated power supply.

10. The cartridge system of claim 8, wherein the supply of the one or more liquid reagents comprises a plurality of containers arranged circumferentially.

11. The cartridge system of claim 8, wherein the supply of the one or more liquid reagents comprises flexible or non-rigid containers.

12. The cartridge system of claim 11, wherein container comprises a spout, and wherein the cartridge further comprises a sealing member for each spout configured to automatically seal the spout.

13. The cartridge system of claim 12, wherein the sealing member comprises an outer wall and an inner wall, wherein the spout is received between the outer wall and the inner wall, and wherein:
i. the outer wall is configured to form a seal the spout; and
ii. the inner wall is configured to form a seal with the spout.

14. A cartridge comprising:
a rigid outer cover defining an internal volume; and
one or more flexible or non-rigid reagent containers within the internal volume, each of the one or more reagent containers configured to store a supply of or more liquid reagents for automatic chemistry analysis of recreational water including swimming pools and spas, wherein the reagent containers are arranged circumferentially.

15. The cartridge of claim 14, wherein the reagent containers comprise a metal or foil laminate.

16. The cartridge of claim 14, wherein the cartridge is self-sealing.

17. The cartridge of claim 14, wherein each reagent container comprises a spout, and wherein the cartridge further comprises a sealing member for each spout configured to automatically seal the spout.

18. The cartridge of claim 17, wherein each sealing member comprises an outer wall and an inner wall, wherein the spout is received between the outer wall and the inner wall, and wherein:
iii. the outer wall is configured to form a seal the spout; and
iv. the inner wall is configured to form a seal with the spout.

19. The cartridge of claim 14, further comprising a base with one or more apertures corresponding to the one or more liquid reagents.

20. The cartridge of claim 14, wherein a height of each reagent container within the internal volume is adjustable.
